(19) United States
(12) Patent Application Publication
(10) Pub. No.: US 2006/0084728 A1
(43) Pub. Date: Apr. 20, 2006

(54) Polymer composites containing keratin

(76) Inventors: Justin R. Barone, Rockville, MD (US); Walter F. Schmidt, Beltsville, MD (US)

Correspondence Address: USDA, ARS, OTT, 5601 SUNNYSIDE AVE, RM 4-1159, BELTSVILLE, MD, 20705-5131, US

(21) Appl. No.: 11/249,794

(22) Filed: Oct. 13, 2005

Related U.S. Application Data
(63) Continuation-in-part of application No. 10/750,464, filed on Dec. 31, 2003.

(51) Int. Cl. C08L 89/00; A61K 47/48

(52) U.S. Cl. 524/10; 524/12; 524/17; 524/21

(57) ABSTRACT

Polymer composites containing keratin and at least one polymer (e.g., polyurethane, polyethylene, polypropylene, polyamide, polyesters, polyvinyl chloride, polylactic acid, polyvinyl alcohol, polycaprolactone, polyhydroxy esther ether, polyhydroxybutyrate, polyhydroxyalkanoates, polystyrene, polycarbonate, polymethyl methacrylate, polybutadiene, polyisoprene, polyurethane, epoxies, or mixtures thereof; wherein polypropylene is not atactic polypropylene). Methods for producing the polymer composites are also provided. The properties of polymers are improved by the dispersion of cleaned, processed keratin material, preferably derived from poultry feathers, in the polymeric material. The keratin material can be in the form of fibers, particles, or large pieces from animal hair, nail, hoof, horn, or avian (e.g., poultry) feathers.

### REFERENCE TO RELATED APPLICATION

This application claims the benefit of U.S. patent application Ser. No. 10/750,464, filed 31 Dec. 2003, which is incorporated herein by reference in its entirety.

### BACKGROUND OF THE INVENTION

The present invention relates to polymer composites containing keratin and at least one polymer (e.g., polyurethane, polyethylene, polypropylene, polyamide, polyesters, polyvinyl chloride, polylactic acid, polyvinyl alcohol, polycaprolactone, polyhydroxy esther ether, polyhydroxybutyrate, polyhydroxyalkanoates, polystyrene, polycarbonate, polymethyl methacrylate, polybutadiene, polyisoprene, polyurethane, epoxies, or mixtures thereof; wherein polypropylene is not atactic polypropylene). Methods for producing the polymer composites are also provided.

Polymer composites presently use synthetic fibers (e.g., glass or carbon fibers), natural fibers (e.g., cellulosic fibers like jute, sisal, flax, hemp, kenaf, wood), and inorganic particles or powders (e.g., talc or silica). Inorganic additives used to make polymer composites can have very high modulus but have a very high density compared to the polymer. This adds significantly to the rigidity and possibly strength of the composite materials but to the weight as well, which is detrimental in applications such as automotive and building materials. Inorganic additives are also brittle, so there is a tendency for them to break during processing and also during recycling. In addition, the high modulus of inorganic additives is abrasive to polymer processing equipment, thus reducing the lifetime of the processing equipment by increasing the rate of machine wear. Organic additives like cellulose have much lower modulus and lower density than inorganic additives, but the modulus of such organic fibers or additives is still higher than the polymer and the density is similar to that of the polymer. Organic additives also often do not contain readily extractable fibers of uniform morphology, thus requiring chemical or biochemical treatments in addition to mechanical treatments to release the fibers. Cellulose-based fibers are typically susceptible to degradation at the temperatures experienced during polymer processing. In addition, some organic fibers, especially those derived from plants, may be susceptible to rapid environmental degradation and are only available seasonally. One advantage of being able to use organic material as an additive in polymer composite formulations is to replace synthetic material derived from petroleum with a low cost, environmentally-friendly material while maintaining current polymer properties. This has proven challenging to attain on a commercial scale.

In fiber reinforced polymer composites, the integrity of the fiber/polymer interface needs to be high for efficient load transfer. Ideally, the molten polymer would spread over and adhere to the fiber, thus creating a strong adhesive bond. Inorganic fibers like glass and cellulosic fibers have hydrophilic surfaces that make them incompatible with hydrophobic polymers. Therefore, inorganic and cellulosic fibers usually require chemical modification to increase fiber/polymer interactions. The chemical modification, using a coupling agent, acts as a “bridge” between the inorganic fiber and the organic polymer. The “bridge” must adhere or bond to the fiber and, in turn, strongly interact with the polymer. When using glass fibers, the coupling agent has a hydrophilic side that is compatible with the fiber and a hydrophobic side that is compatible with the polymer. In glass fibers, the coupling agent reacts with the surface of the glass forming covalent bonds. Without the coupling agent, there is simply adhesion of the polymer to the glass through weak bonding (i.e., van der Waals or induction interactions). Hydrophilic groups on the peptide backbone (e.g. serine, threonine, aspartate) of organic fibers offer chemically distinct sites for covalently bonding the polymer to the fiber either directly or through a similar type of chemical “bridge”. Covalent bonds are much stronger than induction or van der Waals interactions so a covalently bonded interface would be advantageous.

Most studies of naturally occurring organic fibers concentrate on cellulose-based fibers obtained from renewable plant resources such as wood, flax, sisal, or jute. There are very few studies detailing composites made from protein fibers obtained from agricultural resources. We decided to investigate keratin from poultry feathers as a fiber resource since over four billion pounds of chicken feather waste are generated by the U.S. poultry industry each year.

### SUMMARY OF THE INVENTION

Polymer composites containing keratin and at least one polymer (e.g., polyurethane, polyethylene, polypropylene, polyamide, polyesters, polyvinyl chloride, polylactic acid, polyvinyl alcohol, polycaprolactone, polyhydroxy esther ether, polyhydroxybutyrate, polyhydroxyalkanoates, polystyrene, polycarbonate, polymethyl methacrylate, polybutadiene, polyisoprene, polyurethane, epoxies, or mixtures thereof; wherein polypropylene is not atactic polypropylene), and methods for producing the polymer composites.

We have discovered that the properties of polymers are improved by the dispersion of cleaned, processed feather fiber and quill material derived from poultry feathers. In accordance with this discovery, it is an object of the invention to provide novel compositions comprising mixtures of keratin feather fiber and keratin quill material with polymers.

It is also an object of the invention to provide methods of producing the novel keratin/polymer composites.

Other objects and advantages of the invention will become readily apparent from the following description.

### DETAILED DESCRIPTION OF THE INVENTION

The present invention relates to polymer composites containing keratin and at least one polymer (e.g., polyurethane, polyethylene, polypropylene, polyamide, polyesters, polyvinyl chloride, polylactic acid, polyvinyl alcohol, polycaprolactone, polyhydroxy esther ether, polyhydroxybutyrate, polyhydroxyalkanoates, polystyrene, polycarbonate, polymethyl methacrylate, polybutadiene, polyisoprene, polyurethane, epoxies, or mixtures thereof; wherein the term polypropylene does not include atactic polypropylene). Methods for producing the polymer composites are also provided; the methods do not utilize urea, formaldehyde or other chemicals which degrade keratin. In the present invention the properties of polymers are improved by the dispersion of cleaned, processed keratin material, preferably derived from poultry feathers, in the polymeric material. This results in a composite material; the present invention does not concern a polymer blend. The keratin material can be in the form of fibers, particles, or large pieces from animal hair, nail, hoof, horn, or avian (e.g., poultry) feathers; however, the keratin material is not ground into a fine powder (e.g., average particle size of 30 micrometers or less). In composites applications, it is important to maintain what is defined in the art as a high aspect ratio. Aspect ratio refers to the length to diameter ratio of cylindrically shaped fibers or the length to width or length to thickness ratio of rectangularly shaped fillers. Fibers derived from poultry feathers have a well-defined diameter of 5 micrometers. The best composite strength and stiffness properties were obtained when the aspect ratio was maximized; the aspect ratio is preferably greater than about 100 up to about 1000 (e.g., greater than 100 up to 1000), more preferably about 200-about 800 (e.g., 200-800). Feather fiber in its native form has an aspect ratio of 200 to about 1000 so grinding or shortening the fiber would not be preferred from the standpoint of increasing mechanical properties like strength and stiffness because fibers that are long relative to their diameters are capable of carrying larger amounts of applied load. However, where surface appearance may be a highly desirable feature, smaller aspect ratios on the order of about 10 to about 100 (e.g., 10-100) may be desired to optimize strength and stiffness but also allow for aesthetic appeal. Preferably the keratin material has not been subjected to reduction or oxidation agents, nor has it been modified or degraded (e.g., alkaline hydrolysis of keratin through the action of a strong base or through the use of urea, formaldehyde or other chemicals which degrade keratin). The present invention does not utilize polypeptides or peptides derived from keratin; instead the natural protein is utilized.

Polymers which may be used include those known in the art, including synthetic semi-crystalline polymers such as thermoplastic polyurethane, polyethylene, polypropylene, polyamide, polyesters, polyvinyl chloride, and their copolymers; these are the most important polymers commercially and the materials most likely to benefit from the keratin material reinforcement. Polypropylene as used herein is defined as excluding atactic polypropylene. Other synthetic semi-crystalline polymers such as polylactic acid, polyvinyl alcohol, polycaprolactone, polyhydroxy ester ether, polyhydroxybutyrate, polyhydroxyalkanoates, and their copolymers, known as “bioresorbables” because of enhanced degradation tendencies, may also be utilized. Synthetic amorphous polymers such as polystyrene, polycarbonate, and polymethyl methacrylate may also be utilized, though they are physically less likely to benefit from the advantages of keratin reinforcement. Network or cross-linked polymers like polybutadiene, polyisoprene, polyurethane, and epoxies are also likely to benefit greatly from addition of keratin. Polymers that can be derived from agricultural feedstocks would be the ideal materials to mix with keratin to make 100% agricultural and sustainable composites. Polymers that can be obtained from agricultural feedstocks include polylactic acid, polyhydroxyalkanoates, thermoplastic starch, zein protein, wheat gluten, chitosan, and epoxidized soybean polymers. The polymer composite may contain one or more of the above polymers.

Improvements resulting from use of keratin in polymers include increased modulus (or rigidity), strength, toughness or energy to break, biocompatibility/biodegradability/environmental-friendliness, decreased weight or density, increased appearance, and decreased cost. The addition of foaming agents to the keratin/polymer composites produces a very lightweight material with a unique morphology at the microscopic level. There can be isotropic or anisotropic enhancement of material properties relative to the loading axis based on the processing of the composites and subsequent orientation of the keratin material. One surprisingly important feature is that the semi-crystalline keratin material enhances the resistance of polymer-based composites to mechanical stress. The keratin material is a durable, renewable, nontoxic, and environmentally-friendly material incorporated into polymer composite formulations. In principle, these new keratin/polymer composites are easier to recycle. In addition, an added value is also given to the present utilization of the keratin material (e.g., poultry feathers).

As noted above, inorganic additives previously used to make polymer composites can have very high modulus and very high density compared to the polymer. This adds significantly to the rigidity and possibly strength of the composite materials but to the weight as well, which is detrimental in applications such as automotive and building materials. Keratin from feathers is 2.5 times less dense than glass fiber and has a modulus that is 10 times lower. However, normalizing the modulus by the density shows that the modulus to weight ratio of keratin is only 3.5 times lower than that of glass fiber, thus the stiffness to weight ratio or strength to weight ratio is close to glass. Glass is much stronger than feathers, but the strength comes at a large weight cost. Surprisingly when one looks at strength to weight ratio as the performance standard, rather than just strength itself, they are much more comparable.

It is interesting to note that in some applications involving polymer composites, glass, talc, mica, and calcium carbonate are not even added to increase strength or stiffness. Instead, these inorganic materials are added for dimensional stability (usually at the expense of a large weight gain) to combat warpage during the manufacturing process. Polyethylene and polypropylene have high amounts of crystallinity. After molding and cooling, there is a large amount of warpage due to the fast cooling rates used by polymer processors. Fast cooling rates are used by the industry to keep processing times to a minimum and save money, thus cooling at slower rates to avoid warpage is not cost-effective. Surprisingly, using feather fiber instead of inorganic materials commonly used is ideal because it suppresses warpage during cooling, remains intact throughout the process, and does not add significant weight.

Feathers are made of keratin, which contains ordered α-helix or β-sheet structures and some disordered structures. The feather fiber fraction has slightly more α-helix over β-sheet keratin. The clear outer quill has much more β-sheet than α-helix. This leads to a crystalline melting point of ca. 230° C. for the outer quill compared to ca. 240° C. for the feather fiber. Feather keratin has a molecular weight of about 10,700 g/mol and a cysteine/cystine content of 7% in the amino acid sequence. Cysteine is a sulfur containing amino acid responsible for the sulfur-sulfur bonding or cross-linking in the keratin. No chemical or biochemical treatment is required to release keratin from feathers. Keratin material derived from poultry feathers has the unique features of a semi-crystalline and cross-linked structure at the molecular level, as well as higher length scale structures. These features impart a high modulus to the keratin material, at least an order of magnitude higher than the polymer itself, but still an order of magnitude lower than glass or carbon fibers. The density of the keratin material is 0.89 g/cm³, which is equal to or less than the density of the polymer. The keratin is derived from a renewable resource (e.g., feathers) and has the ability to deform slightly during processing, therefore it does not contribute to processing equipment wear. The degradation temperature of the keratin material, approximately 220°-240° C., is higher than the temperature of most polymer processing applications, therefore the keratin material overcomes a lot of the detriments of previously used inorganic materials and organic materials derived from plants. Since the degradation temperature of the keratin material is approximately 220-240° C., preferably the temperature of the keratin/polymer processing is below about 200° C. (e.g., ≦200° C.).

Keratin molecules have a mixed 60% hydrophobic/40% hydrophilic surface chemistry that can be exploited to get them to disperse in and interact with a wide variety of polymers without the use of high forces or expensive chemical agents. The mixed hydrophobic/hydrophilic surface chemistry makes the keratin fibers at least partially compatible with all polymers, which are usually predominantly hydrophilic or hydrophobic. The highest sales volume polymers are polyethylene and polypropylene, both of which are 100% hydrophobic. Inorganic materials and cellulose-based materials are predominantly hydrophilic. Therefore, inorganic and cellulose materials need to undergo a chemical surface modification to make them compatible with the most important commercial polymers. In contrast, keratin fibers generally do not require such chemical modification and can thus be used as a “drop-in” replacement for inorganic materials and cellulose-based materials.

Another advantage of keratin fibers is that they have some surface roughness that gives the fibers more surface area for interaction with polymer molecules (compared to a microscopically smooth fiber). High polymer-fiber adhesion is crucial for effective load transfer throughout the composite.

Keratin material may be prepared from feathers processed into fibers and quill pieces as described in Gassner et al. (U.S. Pat. No. 5,705,030) which is incorporated by reference in its entirety. The keratin feather fiber is cleaned and separated from the quill fraction using the Gassner et al. method which comprises five basic steps: collecting raw feathers, washing the feathers in a water soluble organic solvent, drying the feathers, removing the fiber from the quill shafts, and collecting the fiber and quill fractions. The feather fiber generally has a uniform macroscopic diameter of approximately 0.0005 cm. The density of feather fiber is determined by displacing a known volume and weight of ethanol with an equivalent amount of fiber. A density value of ρ_{f}=0.89 g/cm³ is generally obtained. Fibers of about 0.02 cm or greater length (e.g., about 0.02-about 1 cm) may be made by grinding feather fiber using a Retsch ZM 1000 centrifugal grinder. The preferred range of the length of the fibers would be lengths that maximized aspect ratio (discussed above) for the application so as to maximize strength and stiffness; for feather fiber this would be the intrinsic length scale of the fibers, about 1 mm to about 1 cm. The rotational velocity of the instrument is 15000 rpm and contains a torque feedback so as to not feed in too much material and overload the motor. The fiber is fed in slowly to avoid motor overload and to minimize frictional heating of the instrument and the fiber. Fiber lengths smaller than about 0.02 cm may be made by grinding the fiber on a Retsch PM 400 ball mill. Feather fiber is loaded into 500 ml stainless steel grinding vessels so that it occupies about a quarter of the volume. The grinding media are four 4 cm stainless steel spheres for a total of 1132 g grinding media. Grinding proceeds at 200 rpm for 30 minutes. Each ground fraction is sieved on a Retsch AS 2000 vibratory mill. To achieve good separation, 1.0 cm glass beads are used as sieving aids to aid the separation process. Sieving occurs at a constant frequency concurrent with the AC outlet but amplitude and time can be varied. The material is loaded into the top sieve of the stack. The sieving stack contains eight sieves with hole sizes from 0.0710 cm to 0.0038 cm. Sieving at an amplitude of 1.0 (arbitrary instrument scale) for 60 minutes effectively separates the “fines” from the desired average fiber length.

Feathers from any avian species (e.g., chickens, turkeys) may be utilized because feathers from all avian sources have the characteristics that are necessary for the production of useful fibers. Feathers are made up of many slender, closely arranged, parallel barbs forming a vane on either side of a tapered hollow shaft. The barbs have bare barbules that, in turn, bear barbicels commonly ending in hooked hamuli and interlocking with the barbules of an adjacent barb to link the barbs into a continuous vane.

Structurally, the keratin feather fiber portion has naturally occurring nodes approximately 50 microns apart. These nodes are potential cleavage sites for producing fibers that are uniformly 40 to 50 microns in length. In addition, feathers from different species vary in length: poultry feathers are approximately 2 cm in length while those from exotic birds such as peacocks, emus, or ostriches are 4 to 5 cm or longer. Keratin feather fibers from poultry are also thinner than other natural fibers, with a constant diameter of approximately 5 microns. This makes it possible to produce high aspect (length to diameter) ratio fibers. High aspect ratio fibers are needed to get increased mechanical properties in polymer composites. The keratin quill material can be processed into different sizes and shapes.

The fiber portion of the feather as well as the quill consists primarily of the protein keratin. Although both portions of the feather are made of keratin, each has unique properties. Keratin feather fiber and inner quill material are 41% α-helix, 38% β-sheet, and 21% random (disordered) protein structures. The α-helical structure contains important intra-molecular hydrogen bonds between the amide and carbonyl groups in the protein backbone. The β-sheet structure contains major inter-chain hydrogen bonding between the amide and carbonyl groups in the protein backbone. The hydrogen bonding can be correlated with the bound water in the protein structure. The helices can pack together to form crystals. The semi-crystalline and cross-linked structure in keratin feather fiber results in a polymer with a relatively high elastic modulus of approximately 3.4 to 5 GPa. The outer quill contains 50% α-helix with 20% β-sheet and the balance disordered structures. Differential scanning calorimetry (DSC) shows that outer quill has a crystalline melting temperature of about 230° C. while the feather fiber and inner quill have a melting temperature of about 240° C.

Keratin material/polymer composites ranging from low, e.g., <1 percent by weight or volume, to high, e.g., >99 percent by weight or volume, fractions of keratin material can be made according to the present invention. For instance, keratin percentages from <1% up to 50% by weight or volume are useful to make high performance polymer composites and foams. Keratin percentages greater than 50% by weight or volume are preferred for filters, insulation material, and to make “concentrates” for pelletizing and subsequent use in composite materials.

Keratin fiber has a constant 0.0005 cm diameter. The typical poultry feather fiber length is 2 to 3 cm. Grinding as described previously can produce uniformly sized fibers down to 38 microns. Smaller fibers are possible with the proper equipment. If it is desired to increase strength or stiffness, the aspect (length to diameter) ratio of the fiber needs to be high. For keratin feather fiber, we have determined that the maximum physical properties are obtained at aspect ratios of 50 or greater. This is concurrent with the value for many other fibers. The aspect ratio can be smaller than this but strength and stiffness will not be maximized.

The quill material is in long continuous strips typically of about 1-10 cm length and 0.1-1 cm diameter. This material can be ground to a multitude of sizes depending on the grinding application employed. For instance, after disconnecting the quill from the feather fiber in the process as outlined in U.S. Pat. No. 5,705,030, the quill is in pieces that are about 1 cm long and 0.5 cm wide. The lack of one uniform dimension, like the uniform diameter of the fiber, makes it difficult to achieve high aspect ratios with chopped quill material and therefore large physical property increases when made into polymer composites. However, the low aspect ratio of the chopped quill material allows it to be a perfect additive to fight warpage during fast mold cooling cycles in processing. In addition, the chopped quill material looks very much like chopped wood fiber frequently used in wood products. So addition of chopped quill material to polymers allows them to look similar to particle board wood products. It is anticipated that the keratin/polymer composites could be made using any polymer processing technique. Currently, the most popular polymer processing techniques are extrusion and injection molding, but it is anticipated that the composites could be made on roll-mills, batch mixers, compression-molders, vacuum formers, or any other of the typical polymer processing techniques. We have found that the keratin can be processed for any length of time at 200° C. or below. At temperatures of 200°-210° C., the keratin is stable for about 2 to 4 minutes and at temperatures of 210-220° C. the keratin is stable for 2 minutes or less. Many polymer processing techniques occur within two minutes so it is possible to process keratin up to 220° C. In our laboratory, keratin material ground up as previously described was mixed with polymers at keratin contents of 1% up to 90%. Mixing was performed on a Brabender co-rotating screw mixing head or a Brabender 25:1 single screw extruder at temperatures from 150° C. to 220° C. using velocities up to 200 rpm and times from 2 minutes up to several hours, depending on stability. Molding of keratin/polymer composites in our laboratory was conducted on a Carver Press Autofour/30 Model 4394 at pressures of 3000 N up to 260,000 N and temperatures of 150-220° C. for various times, depending on stability. Additives such as foaming agents, mordants, and dyes (e.g., titanium dioxide and iron oxide), binders (e.g., starch and casein), hardeners, chemical sizing or coupling agents (e.g., a ketene dimer emulsion, titanates, maleated polyolefins, and silanes), fillers and plant fibers (e.g., kenaf, cotton rag, wood cellulose) or animal fibers (e.g., collagen) may be included in the composites. These additives are well known to those skilled in the art and can be varied according to the requirements for the ultimate product. Chemical cross-linking, wetting, and/or redox reagents may also be utilized as needed. For highly crystalline polymers like high density polyethylene and polypropylene, coupling agents are critical to prevent the polymer from contracting off of the fiber surface upon cooling.

Processes that are well known in the art may be used to manufacture the polymer composites. In the examples below we utilized two major processing techniques to manufacture prototype plastic samples: solvent casting and polymer melt blending. Processing is optimized to retain critical molecular and morphological properties of the keratin.

In solvent casting, the keratin material is dispersed in a solvent in which the polymer is soluble and the keratin is not soluble. The keratin material is added to the polymer with mechanical or instrumental mixing such as sonication, internal mixing, or extrusion, with or without heating. The resultant composite material is formed or molded into the desired end product shape and the solvent is removed by heat and/or vacuum. Depending on the mixing and forming technique used, multiple morphologies are possible that may have isotropic or anisotropic properties. Useful solvents known in the art include but are not limited to water, ethanol, methyl ethyl ketone, acetone, and toluene.

In polymer melt blending, keratin material is mixed into molten polymer using an internal mixer, extruder, or mill. The resultant composite material is injection molded, blow molded, cast, or extruded with standard equipment known in the art. The process may be multi-step. The initial steps may involve the dispersion of a large volume of keratin material into a small volume of low viscosity polymer (referred to as “polymer one”) to make a keratin material “concentrate”. Concentrates are commonly used by composites manufacturers. The reason to make a concentrate is to allow for easy bulk handling of the keratin material. Most composites manufacturers are not equipped to handle bulk fibers and additives. However, providing the fiber or additive in a concentrate (“pellet”) form makes them universally usable by the composites industry, which is equipped to handle polymers. “Polymer one” should be compatible with the polymer being used by the composites manufacturer (referred to as “polymer two”). The reason for the low viscosity of “polymer one” is that the addition of fibers or additives increases the overall viscosity of the concentrate and the viscosity of the concentrate must be approximately equal to the viscosity of “polymer two” for easy dispersion. So the secondary steps involve mixing the keratin concentrate into a bulk molten polymer or “polymer two”. The processes may also be combined for the manufacture of a single product.

Molded articles may be prepared from the polymer composite using methods and equipment known in the art.

Specific examples of effective processes are described below. The following examples are intended only to further illustrate the invention and are not intended to limit the scope of the invention as defined by the claims. Feather fiber processed as described in Gassner et al. (U.S. Pat. No. 5,705,030) was used in all examples.

### EXAMPLES

### Example 1

Approximately 10 percent by weight of polystyrene (PS) (Aldrich Chemical, M_{w}=240,000, M_{w}/Mₙ=1.71, T_{g}=94° C., MFI=7.5 g/10 min @ 200° C./5 Kg, ρ=1.047 g/cm³) was dissolved in acetone to produce a material with a “dough-like” consistency. Approximately 20 percent by weight of feather fiber, processed as per U.S. Pat. No. 5,705,030, was added to the polystyrene/acetone “dough” as was a small amount of Pigment Red-101 iron oxide (Carriage House Paper) to add color. The polystyrene/acetone/feather fiber/pigment system was then sonicated for 15 minutes (45 seconds on/15 seconds off sequence) at 35 watt power using a Misonix Sonicator 3000 to equally disperse all materials. The resulting material was further processed by rolling it with an aluminum bar on a flat surface. This is a similar operation to “milling” commonly encountered in polymer processing where large extensional deformations are imparted to the material to enhance dispersion. The material can now be filled into a mold and the acetone evaporated slowly or quickly. Evaporating the acetone slowly resulted in a dense, non-porous structure on a macroscopic scale, while evaporating the acetone quickly resulted in a foamed, porous structure on a macroscopic scale. The modulus of the feather fiber allows it to bend around the air bubbles, thus reinforcing them, indicating an interesting morphology that can only be attained under the correct conditions. Pores from air bubbles, although lowering material weight, are weak points in the structure. When the fibers surround the pores, they protect them from load. Stiffer fibers, like glass, do not bend around the pores as the air bubbles form and grow. The stiff fibers would then protrude into the pores and become weak points also. Testing was performed using Com-Ten 95 RC Series Tensile Tester in three-point bending mode. Compared to commercially available “chip board” wood product, the foamed polystyrene/feather fiber material has a similar value of modulus or rigidity, a density that is 12% lower, and a yield strength that is, on average, 2 to 3 times higher.

### Example 2

Feather fiber that was processed as described in U.S. Pat. No. 5,705,030 was passed through a Retsch Ultracentrifuge Mill ZM 1000 containing a 0.2 mm screen. Therefore, the average feather fiber length was 0.2 mm. Feather fiber has a relatively constant diameter of 0.005 mm yielding an average feather fiber aspect ratio of 40. Feather fiber weight fractions of 0%, 5%, and 10% were mixed with Dowlex 2037 linear low density polyethylene (LLDPE) (Tₘ=124° C., MFI=2.5 g/10 min @ 190° C./2.16 Kg, ρ=0.935 g/cm³) from Dow Chemicals. Mixing was performed “by hand” in the following manner: The LLDPE was placed in a metal beaker and then in a convection oven set to 160° C. The temperature of the LLDPE inside the metal beaker was monitored with a thermocouple. Upon reaching 160° C., the beaker was removed from the oven and the feather fiber added and mixed with a metal spatula until the polyethylene had cooled enough to solidify. The LLDPE/feather fiber system was then placed back in the oven and the temperature of the melt again monitored. Upon reaching 160° C., the metal beaker was removed from the oven and the mixing process repeated. The total mixing time was one hour to get good dispersion, assessed visually, of the feather fiber in the LLDPE. The LLDPE/feather fiber material was in the oven approximately half of the total mixing time. For the system with no feather fiber, the exact same mixing process was performed.

The processed LLDPE and LLDPE/feather fiber was then pressed in a Carver Press Autofour/30 Model 4394 at 170° C. between Teflon-coated aluminum sheets using an applied pressure of 44,482 N. The first press was without shims between the Teflon sheets and the pressure was removed immediately after attaining the 44,482 Newton pressure. Two subsequent presses were performed using 0.76 mm thick shims for approximately 30 seconds each to yield a sheet with a uniform thickness. Tensile bars similar to the Type IV configuration given in ASTM D638 were cut from the composite material sheet. Testing was performed using Com-Ten 95 RC Series Tensile Tester. Some tensile bars were tested immediately and some were tested the next day. The applied speed of the tensile test was 117 mm/min. A clear increase in the elastic modulus was observed in the composite materials over the virgin LLDPE material. For five weight percent 0.2 mm feather fiber there was an average increase of elastic modulus of 12%. For 10 weight percent 0.2 mm feather fiber there was an average elastic modulus increase of 19%. This modulus increase persisted after testing the next day, except the absolute magnitude of modulus for each sample increased about 4%, presumably due to attainment of a solid-state crystal structure. This time-dependent effect has been observed for semi-crystalline polymers. There was no significant change in the density of the composites over that of the virgin polymer.

### Example 3

Feather fiber that was processed as described in U.S. Pat. No. 5,705,030 was passed through a Retsch Ultracentrifuge Mill ZM 1000 containing a 1.0 mm screen. Therefore, the average feather fiber length was 1.0 mm. Feather fiber had a relatively constant diameter of 0.005 mm yielding an average feather fiber aspect ratio of 200. Feather fiber weight fractions of 0% and 0.5% were mixed with a very low density thermoplastic elastomer product from Dow-DuPont elastomers called Engage. The Engage product has a low value of polydispersity, M_{w}/Mₙ˜2, because it was polymerized with a metallocene catalyst. In addition, the Engage product had a density of ca. 0.87 g/cm³ in the solid-state and a melting transition temperature of ca. 60° C. The viscosity of the Engage product was ca. 400 Poise at 180° C. This made the material ideal to mix “by hand”. Mixing was performed the same way as described in Example 2 with the exception that it took half as long. This was presumably because of the lower viscosity of the Engage polymer.

The processed polymer/feather fiber was then pressed in a Carver Press Autofour/30 Model 4394 at 100° C. between Teflon-coated aluminum sheets using an applied pressure of 177,928 N. Only one press was performed using 0.76 mm thick shims for a period of 30 seconds. This yielded a sheet with a uniform thickness. Tensile bars similar to the Type IV configuration given in ASTM D638 were cut from the composite material sheet. Testing was performed using Com-Ten 95 RC Series Tensile Tester. Some tensile bars were tested the same day, some were tested 4 days later, and some were tested 43 days later. The applied speed of the tensile test was 343 mm/min. A clear 11% increase of the elastic modulus was observed in the composite materials over the virgin polymer material. The Engage polymer did not show an increase in the elastic modulus as a function of testing time like the LLDPE Dowlex 2037 material. This was presumably because of the much lower crystallinity in the Engage polymer. There was no significant change in the density of the composite over that of the virgin polymer.

### Example 4

Feather fiber that was processed as described in U.S. Pat. No. 5,705,030 was passed through a Retsch Ultracentrifuge Mill ZM 1000 containing screens with hole sizes of 0.2, 1.0, and 2.0 mm, which corresponded to the major fiber length. Sieving analysis using a Retsch AS2000 Vibratory Sieving apparatus revealed that more than 98% of the total fiber content was similar to the screen size with 2% being fines. In addition, a feather fiber “powder” was made by grinding feather fiber in a Retsch PM 400 Ball Mill using various grinding media at 200 rpm for 30 minutes. The powder obtained had a distribution of sizes with the majority of the fibers, 83%, being 53 microns in length. Feather fiber had a relatively constant diameter of 0.005 mm. Therefore, the “aspect ratio” or ratio of length to diameter, a critical feature to property enhancement in composites, was varied. Feather fiber weight fractions of 0% to 50% were mixed with a commercially available low density polyethylene (LDPE) polymer from Dow chemicals called LDPE LD133A. The LDPE has a viscosity of about 4×10⁶ Poise at 180° C. and a solid-state density of 0.923 g/cm³. Mixing was performed on a Brabender mixing head set at 150° C. Mixing proceeded for 10 minutes at 50 rpm and the actual recorded melt temperature was 180° C. because of the viscous dissipation of the polymer melt during mixing.

The mixed polymer/feather fiber was then pressed in a Carver Press Autofour/30 Model 4394 at 160° C. between Teflon-coated aluminum sheets using an applied pressure of 133,446 N for 18 seconds. This was done three times to yield three films ca. 0.254 mm in thickness. The state of keratin fiber dispersion was then assessed. These three thin films were stacked on one another and pressed for 2 minutes at 8,896 N to yield a film of uniform 3 mm thickness. Tensile bars similar to the Type IV configuration given in ASTM D638 were cut from the composite material sheet. Testing was performed using Com-Ten 95 RC Series Tensile Tester one week later. The applied speed of the tensile test was 127 mm/min.

Over a keratin feather fiber loading range of 0 to 50 weight percent, the elastic modulus of the LD133A-based composites increased 3 times and the yield stress increased 2.5 times while the density decreased 2%.

All of the reported increases are outside of experimental error.

### Example 5

Although individual feather fiber has a density comparable to a lot of fluids (0.89 g/cm³), the bulk density of an agglomeration of fibers is much lower. This makes the fiber agglomerates difficult to handle. Dispersing the agglomerates into fluids allows them to be handled with ease. For instance, it is typical to disperse fibers or fillers in low viscosity fluids at high loadings, i.e., greater than 90 weight percent fiber. This is known in industry as a “concentrate” and is the standard way to package and sell fibers and fillers. The concentrate can then be mixed with, for instance, polymers to make composites, or aqueous solutions of alcohols to make consumer products such as cosmetics, etc.

A flat-bottomed beaker 10 cm in diameter and 8 cm high was used as the cell for dispersion experiments at room temperature. The beaker was filled with 200 ml of the dispersing fluid and placed on a stirring plate. A 6.4 cm long Teflon-coated stirring bar with hexagonal cross-section (0.95 cm wide) was used to generate the flow. Angular fluid velocity, co, was measured by placing colored tracer particles approximately 0.1 cm in diameter into the moving fluid and noting the speed along the circumference of the beaker in revolutions per second. A keratin feather fiber agglomerate was added to the moving fluid and the dispersion of the individual feathers monitored as a function of time with a digital camera. Initially, the keratin fibers adhered to one another forming a large agglomerate. The fiber agglomerates had low bulk densities, <0.01 g/cm³.

Alkanes such as hexane contain only carbon and hydrogen (CH) and are 100% hydrophobic. Alcohols such as methanol or ethanol contain oxygen and hydrogen (OH) groups that add hydrophilicity to the molecule. Short-chain alcohols are predominantly hydrophilic. Keratin has 40% hydrophilic and 60% hydrophobic chemical groups as well as a certain amount of hydrogen-bound water in the structure.

100% fiber dispersion was observed after short times and low applied hydrodynamic forces in hydrophilic fluids like ethanol and methanol. Partial fiber dispersion was observed after very long times, i.e., hours, over all applied hydrodynamic forces for hydrophobic fluids like hexane. This indicated that dispersion of keratin fiber was maximized in hydrophilic materials.

### Example 6

Feather fiber that was processed as described in U.S. Pat. No. 5,705,030 was passed through a Retsch Ultracentrifuge Mill ZM 1000 containing a screen with a hole sizes 1.0 mm, which corresponded to the major fiber length. Ten weight percent of the 1.0 mm long keratin feather fiber was dispersed in high density polyethylene (HDPE) from Exxon known as HDPE 7760 and a copolymer of vinyl alcohol and 44% ethylene (PVA-co-PE) from Aldrich. The HDPE is a purely hydrophobic polymer and the PVA-co-PE is a predominantly hydrophilic polymer. The HDPE has MFI=0.06 g/10 min at 190° C. and the PVA-co-PE has MFI=3.5 g/10 min at 210° C. The melting temperatures of HDPE 7760 and PVA-co-PE are 129° C. and 165° C., respectively. The HDPE has a solid-state density of 0.952 g/cm³ and the PVA-co-PE has a solid-state density of 1.2 g/cm³. Dispersion occurred in a Brabender mixing head at 180° C. and 50 rpm for 10 minutes at a 60% degree of fill. The actual melting temperature was 200° C. because of the viscous dissipation of the polymer melt during processing. To check the state of dispersion, material was removed from the mixer periodically and the filled polymers were pressed on a Carver Press Autofour/30 Model 4394 at 180° C., 133,446 N for 18 seconds. Complete dispersion of the feather fiber was observed after about 6 minutes. This indicated that the high hydrodynamic forces encountered during polymer processing were enough to overcome the lack of chemical compatibility, or, the keratin loses a large amount of bound water from its structure, thus making it compatible with hydrophobic polymers.

### Example 7

Feather fiber that was processed as described in U.S. Pat. No. 5,705,030 was passed through a Retsch Ultracentrifuge Mill ZM 1000 containing a screen with a hole size of 1.0 mm, which corresponded to the major fiber length. Sieving analysis using a Retsch AS2000 Vibratory Sieving apparatus revealed that more than 98% of the total fiber content was similar to the screen size with 2% being fines. In addition, a feather fiber “powder” was made by grinding feather fiber in a Retsch PM 400 Ball Mill using various grinding media at 200 rpm for 30 minutes. The powder obtained had a distribution of sizes with the majority of the fibers, 83%, being 53 microns in length. Feather fiber had a relatively constant diameter of 0.005 mm. Therefore, the “aspect ratio” or ratio of length to diameter, a critical feature to property enhancement in composites, was varied. The 53 micron fiber had an aspect ratio of 10.6 and the 1 mm fiber had an aspect ratio of 200. Feather fiber weight fraction was kept constant at 20 weight percent and was mixed with a commercially available high density polyethylene polymer from Exxon Chemicals referred to as HDPE 7760. The HDPE had MFI=0.06 g/10 min at 190° C. and a melting temperature of 129° C. The HDPE had a solid-state density of 0.952 g/cm³. Mixing was performed on a Brabender mixing head at temperature settings from 140° C. to 200° C. These corresponded to actual mixing temperatures of ca. 165° C. to 225° C. because of the viscous dissipation of the polymer melt during mixing. Mixing proceeded at 50 rpm for times from 1 to 30 minutes. These samples were then pressed in a Carver Press Autofour/30 Model 4394 at 160° C. between Teflon-coated aluminum sheets using an applied pressure of 133,446 N for 18 seconds. This yielded films ca. 0.254 mm in thickness. The state of keratin fiber dispersion was then assessed. The thin films were then pressed for 3 minutes at 8,896 N to yield a film of uniform 3 mm thickness. Tensile bars similar to the Type IV configuration given in ASTM D638 were cut from the composite material sheet. Testing was performed using Com-Ten 95 RC Series Tensile Tester one week later. The applied speed of the tensile test was 127 mm/min.

This analysis showed the optimum mixing conditions for keratin feather fiber/polyethylene composite materials. Increases in composite modulus of 30% over the virgin polymer were found when mixing at temperatures of 190° C. or less for 15 minutes or less. High dispersion (nearly 100%) of keratin fibers was found between 10 and 15 minutes of mixing.

To study the effect of molding on keratin feather fiber/HDPE composites, a large amount of 20 weight percent composite material was mixed at 50 rpm, a set temperature of 140° C. (actual temperature is 170° C.), and 15 minutes. The fiber dispersion was checked by pressing thin films of material in the Carver Press at 160° C. between Teflon-coated aluminum sheets using an applied pressure of 133,446 N for 18 seconds. This yielded films ca. 0.254 mm in thickness.

The limits of molding were tested by compression-molding in the Carver Press at 200° C. for times from 2 to 7 minutes and at 220° C. from 0.75 to 2.5 minutes using an applied force of 22,441 N to yield films of uniform 3 mm thickness. Shims 3 mm in thickness were used during the compression-molding, which occurred between Teflon-coated aluminum foil sheets. Tensile bars similar to the Type IV configuration given in ASTM D638 were cut from the composite material sheet. Testing was performed using Com-Ten 95 RC Series Tensile Tester one week later. The applied speed of the tensile test was 127 mm/min.

Compression-molding at 200° C. for up to 6 minutes resulted in composite modulus increases of about 20% over the virgin HDPE polymer. Longer molding times resulted in keratin feather fiber degradation and subsequent loss of physical properties. Molding times of 6 minutes were much longer than typical industrial molding operations. Compression-molding at 220° C. resulted in a loss of composite modulus over all molding times, indicating the high temperature of 220° C. caused too much fiber degradation. Compounding or mixing at lower temperatures will completely surround the keratin feather fiber with hydrophobic polymer. When the molding step was introduced, it was anticipated that the fiber would retain water slightly longer because it was surrounded by hydrophobic polymer. It was the intrinsic water content that accounted for the thermal stability of the keratin. The short times of typical molding operations usually mean that a non-steady state temperature exists in the mold. Therefore, there is a high probability that the keratin feather fiber was stable enough to survive most commodity polymer processing operations.

All of the references cited herein are incorporated by reference in their entirety. Also incorporated by reference in their entirety are the following references: Chawla, K. K., Composite Materials, Springer-Verlag 1987; Vincent, J., Structural Biomaterials, Princeton University Press 1990; Lundquist L., et al., Comp Sci Tech, 2003, 63:137-152; Colom X., et al., Comp Sci Tech, 2003, 63:161-169; Jana, S. C., Prieto, A., J Appl Pol Sci, 2002, 86:2159-2167; Jana S. C., Prieto, A., J Appl Pol Sci, 2002, 86:2168-2173; Nunez, A. J., et al., Pol Eng Sci, 2002, 42:733-742; Oksman, K., Clemons, C., J Appl Pol Sci, 1998, 67:1503-1513; Schneider, J. P., et al., J Vinyl Add Tech, 1995, 1:103-108; Maldas, D., et al., J Therm Comp Mat, 1995, 8:420-434; Hughes, M., et al., J Mat Sci, 2002, 37:4669-4676; Jayaraman, K., Comp Sci Tech, 2003, 63:367-374; Rana, A. K., et al., Comp Sci Tech, 2003, 63:801-806; Madera-Santana, T. J., et al., Pol Comp, 2002, 23:991-1002; Parkinson, G., Chem Eng, 1998, 105(3):21; Schmidt, W. F., Jayasundera, S., Microcrystalline keratin fiber, Natural Fibers Plastics, and Composites-Recent Advances, ed. F. Wallenberger and N. Weston, Kluwer Academic Publishers, 2003; Schmidt, W. F., Line, M, J., Physical and chemical structures of poultry feather fiber fractions in fiber process development, TAPPI Proceedings: 1996 Nonwovens Conference, p. 135-140; Fraser R. D. B., et al., Keratins: Their Composition, Structure, and Biosynthesis, Charles C. Thomas Publisher, 1972, p. 31; Murayama-Arai, K., et al., Eur J Biochem, 1983, 132:501-507; Fraser, R. D. B., MacRae, T. P., Molecular structure and mechanical properties of keratins, Symposia of the Society for Experimental Biology Number XXXIV: The Mechanical Properties of Biological Materials, Cambridge University Press, 1980, p. 211-246; Purslow, P. P., Vincent, J. F. V., J Exp Biol, 1978, 72:251-260; Ali, R., et al., J Appl Pol Sci, 2003, 88:1637-1642; Feughelman, M., Mechanical Properties and Structure of Alpha-Keratin Fibres, University of New South Wales Press, 1997; “Definitions Of Terms Related To Polymer Blends, Composites, And Multiphase Polymeric Materials (IUPAC Recommendations 2004)” (Pure Appl. Chem., vol. 76, no. 11, pages 1985-2007 (2004)). Also incorporated by reference in their entirety are the following U.S. patent applications: Ser. No. 10/805,558 filed 19 Mar. 2004; Ser. No. 11/192,907 filed 29 Jul. 2005.

Thus, in view of the above, the present invention concerns (in part) the following:

A composition comprising (or consisting essentially of or consisting of) keratin and at least one polymer.

A polymer composite comprising (or consisting essentially of or consisting of) at least one polymer having dispersed therein keratin.

A polymer composite (polymeric composite) comprising (or consisting essentially of or consisting of) keratin and at least one polymer (polymer matrix).

A polymer composite (polymeric composite), said polymer composite (polymeric composite) comprising (or consisting essentially of or consisting of) keratin and at least one polymer (polymer matrix).

The above polymer composite, wherein said polymer is selected from the group consisting of polyurethane, polyethylene, polypropylene, polyamide, polyesters, polyvinyl chloride, polylactic acid, polyvinyl alcohol, polycaprolactone, polyhydroxy esther ether, polyhydroxybutyrate, polyhydroxyalkanoates, polystyrene, polycarbonate, polymethyl methacrylate, polybutadiene, polyisoprene, polyurethane, epoxies, and mixtures thereof.

The above polymer composite, wherein said polymer is selected from the group consisting of polyurethane, polyethylene, polypropylene, polyamide, polyesters, polyvinyl chloride, and mixtures thereof.

The above polymer composite, wherein said polymer is selected from the group consisting of polyethylene, polypropylene, and mixtures thereof.

The above polymer composite, wherein said polymer is polyethylene.

The above polymer composite, wherein said polymer is polypropylene.

The above polymer composite, wherein said polypropylene is not atactic polypropylene.

The above polymer composite, wherein said keratin has not been subjected to reduction or oxidation agents.

The above polymer composite, wherein said polymer is hydrophobic.

The above polymer composite, wherein said keratin is present in an amount from about 0.1 weight percent to about 99.9 weight percent by weight or volume.

The above polymer composite, wherein said keratin is obtained from a source selected from the group consisting of hair, nail, hoof, horn, avian feathers, and mixtures thereof.

The above polymer composite, wherein said keratin is obtained from avian feathers.

The above polymer composite, wherein said keratin is obtained from a source selected from the group consisting of avian feather fiber keratin, avian feather quill keratin, and mixtures thereof.

The above polymer composite, wherein said keratin is avian feather fiber keratin.

The above polymer composite, wherein said avian feather fiber keratin has an aspect ratio of greater than about 100 to about 1000.

The above polymer composite, wherein said avian feather fiber keratin has an aspect ratio of about 200 to about 800.

The above polymer composite, wherein said avian feather fiber keratin has an aspect ratio about 10 to about 100.

The above polymer composite, wherein said keratin has an average particle size of greater than 30 micrometers (e.g., greater than 300 micrometers).

The above polymer composite, wherein said polymer composite does not contain atactic polypropylene.

The above polymer composite, wherein said avian feather fiber keratin is produced by a method comprising (a) collecting raw feathers, (b) washing, said feathers in a polar water soluble organic solvent, (c) carring out an additional washing step, (d) removing solvent from said feathers, and (e) removing fibers from the feather shafts of said feathers. Wherein said washing step b) is carried out for about one hour. Wherein said polar water soluble organic solvent is ethanol. Wherein said ethanol is about 95% ethanol. Wherein said washing step b) is carried out at a ratio of about 1.0 to about 1.5 gallons of said polar water soluble organic solvent to one pound of feathers. Wherein said polar water soluble organic solvent additionally comprises a surfactant. Wherein said washing step c) is carried out in polar water soluble organic solvent (e.g., ethanol such as about 70% ethanol, or a bactericidal agent such as sodium azide solution). Wherein said washing step c) is carried out in a mixture of ethanol and bactericidal agent. Wherein said washing step c) is carried out at a ratio of about 1.0 to about 1.5 gallons of solvent to one pound of feathers. Wherein said solvent removal step is carried out by heating said fibers at about 60° C. to about 120° C. Wherein said fibers are removed from feather shafts by mechanical shredding or shearing. Wherein said method additionally comprises a step of separating fiber material from shaft material after the removal of fibers from shafts. Wherein said method additionally comprises a step wherein the fibers are subjected to treatment (e.g., beating or chemical treatment with a redox reagent such as hydrogen peroxide and water) to render them soft and pliable, said additional step being carried out subsequent to removal of the fibers from the feather shafts or subsequent to separation of the fibers from the shaft particles. Wherein said fibers are removed from feather shafts by grinding mulching or chopping resulting in a mixture of feather fiber and shaft particles; wherein said method additionally comprises a step of separating feather fibers from shaft particles; wherein said separating step is carried out using a linter, a cone separator, an organ separator or a comb/brush separator or wherein said separating step is carried out using a combination of at least two of a linter, a cone separator, an organ separator and a comb/brush separator linked together by attaching the outlet from a first separator to the inlet of a following separator; wherein said method additionally comprises treating the fibers-to soften them; wherein said treating step is beating or chemical treatment with a redox reagent (e.g., hydrogen peroxide and water).

A molded article prepared from the above polymer composite.

We claim:

1. A polymer composite comprising keratin and at least one polymer selected from the group consisting of polyurethane, polyethylene, polypropylene, polyamide, polyesters, polyvinyl chloride, polylactic acid, polyvinyl alcohol, polycaprolactone, polyhydroxy esther ether, polyhydroxybutyrate, polyhydroxyalkanoates, polystyrene, polycarbonate, polymethyl methacrylate, polybutadiene, polyisoprene, polyurethane, epoxies, and mixtures thereof; wherein said polypropylene is not atactic polypropylene.

2. The polymer composite according to claim 1, wherein said polymer is selected from the group consisting of polyurethane, polyethylene, polypropylene, polyamide, polyesters, polyvinyl chloride, and mixtures thereof.

3. The polymer composite according to claim 1, wherein said polymer is selected from the group consisting of polyethylene, polypropylene, and mixtures thereof.

4. The polymer composite according to claim 1, wherein said polymer is polyethylene.

5. The polymer composite according to claim 1, wherein said polymer is polypropylene.

6. The polymer composite according to claim 1, wherein said keratin is present in an amount from about 0.1 weight percent to about 99.9 weight percent by weight.

7. The polymer composite according to claim 1, wherein said keratin is obtained from a source selected from the group consisting of hair, nail, hoof, horn, avian feathers, and mixtures thereof.

8. The polymer composite according to claim 1, wherein said keratin is obtained from avian feathers.

9. The polymer composite according to claim 1, wherein said keratin is obtained from a source selected from the group consisting of avian feather fiber keratin, avian feather quill keratin, and mixtures thereof.

10. The polymer composite according to claim 9, wherein said keratin is avian feather fiber keratin.

11. The polymer composite according to claim 8, wherein said polymer composite is made at a temperature below the degradation temperature of said keratin or said polymer composite is made in a solvent in which said polymer is soluble and said keratin is insoluble.

12. The polymer composite according to claim 11, wherein said polymer composite is made at a temperature below about 200° C.

13. The polymer composite according to claim 8, wherein said keratin has a molecular weight of about 10,700 g/mol.

14. The polymer composite according to claim 10, wherein said avian feather fiber keratin has an aspect ratio of greater than about 100 to about 1000.

15. The polymer composite according to claim 8, wherein said avian feather fiber keratin has an aspect ratio of about 200 to about 800.

16. The polymer composite according to claim 10, wherein said avian feather fiber keratin has an aspect ratio about 10 to about 100.

17. The polymer composite according to claim 1, wherein said keratin has not been subjected to reduction or oxidation agents.

18. A molded article prepared from the polymer composite according to claim 1.
